(19) United States
(12) Patent Application Publication
(10) Pub. No.: US 2007/0142396 A1
(43) Pub. Date: Jun. 21, 2007

(54) Treatment of pain with 1-[3-[4-(3-chlorophenyl)-1-piperazinyl]propyl]-5-methoxy-3,4-dihydro-2(1H)-quinolone or salt thereof

(75) Inventors: Steve Leventer, Langhorne, PA (US); Robert F. Kucharik, Glenmoore, PA (US); Jeff Calcagno, Hopewell, NJ (US)

Correspondence Address: DRINKER BIDDLE & REATH;ATTN: INTELLECTUAL PROPERTY GROUP, ONE LOGAN SQUARE, 18TH AND CHERRY STREETS, PHILADELPHIA, PA, 19103-6996, US

(73) Assignees: Vela Pharmaceuticals, Inc., Ewing, NJ (US); Otsuka Pharmaceutical Co., Ltd., Tokyo (JP)

(21) Appl. No.: 11/305,593

(22) Filed: Dec. 16, 2005

(51) Int. Cl. A61K 31/496

(52) U.S. Cl. 514/253.07

(57) ABSTRACT

Therapeutic methods are provided for treating pain and fibromyalgia by administration of 1-[3-[4-(3-chlorophenyl)-1-piperazinyl]propyl]-5-methoxy-3,4-dihydro-2(1H)-quinolone or a prodrug or salt thereof.

### FIELD OF THE INVENTION

The present invention relates to methods of treating pain and pain-related disorders, and treatment of fibromyalgia and chronic fatigue syndrome.

### BACKGROUND OF THE INVENTION

Certain carbostyryil derivatives are described in U.S. Pat. Nos. 5,556,857 and 5,656,633. Among the disclosed compounds are 1-[3-[4-(3-chlorophenyl)-1-piperazinyl]propyl]-5-methoxy-3,4-dihydro-2(1H)-quinolone and salts thereof. The monomethanesulfonate, known as “OPC-14523”, has been used as an antidepressant. The aforesaid carbostyryil derivatives are not known for the treatment of pain.

Pain and Pain-Related Disorders

Pain may be nociceptive or neuropathic. According to James L. Hallenbeck, M. D., Palliative Care Perspectives, Oxford University Press, 2003, nociceptive pain results from tissue damage. Intact neurons report damage, and pain is experienced. Nociceptive pain can be subdivided into somatic and visceral (gut) pain. There may be radiation of nociceptive pain, especially visceral pain, but it will not be in a direct nerve distribution. Nociceptive pain is generally responsive to NSAIDs (nonsteroidal anti-inflammatory drugs) and opioids. Conditions associated with inflammation, bone pain, and joint disease are particularly responsive to NSAIDs. Nociceptive pain represents the healthy pain response to thermal and mechanical challenges that might result in tissue damage to an organism. The healthy nociceptive response requires proper functioning of an intact nervous system.

Neuropathic pain, in contrast to nociceptive pain, comprises a perturbation of pain signaling pathways resulting from electrophysiological instability which may be caused by injury to nerve tissue (Summer, Curr. Opin. Neurol., Oct., 16(5): 623-628, 2003; Krarup, Curr. Opin. Neurol., Oct., 16(5): 603-612, 2003. Neuropathic pain occurs when the peripheral and/or central nervous systems are sensitized following an injury to the peripheral system. This initial injury can occur from a wide variety of causes including traumatic physical injury as well as systematic diseases such as diabetes, herpes zoster, AIDS/HIV, syphilis and various other autoimmune diseases. Neuropathic pain serves no protective purpose but persists following viral infection, trauma, certain medications, or metabolic insults.

Central or peripheral nerve tissue damage may result in heightened sensitivity to non-noxious stimuli, and/or an exaggerated response to mild to moderately noxious stimuli. A simple focal peripheral nerve injury may initiate a range of peripheral and central nervous system processes that may contribute to persistent pain and abnormal sensation. Dworkin et al., Arch. Neurol., November; 60:1524-34, 2003.

The manifestation of neuropathic pain may comprise a number of positive and negative symptoms (Loser et al., editors, Bonica's Management of Pain, 3rd ed., Philadelphia, Pa., Lippincott, Williams and Wilkins, 2000). Positive sensory phenomena relate to the exaggerated perception of stimuli (allodynia, hyperalgesia, hyperpathia), wherein application of modest stimuli causes the false perception of a disproportionately large stimuli. Positive motor symptoms include increased muscle tone, tremor, dystonia, and dyskinesia. Negative sensory phenomena include an inappropriate response to light touch, vibration, joint position, pin prick, or warm/cold application to the affected region. Negative motor symptoms include hypotonia, decreased muscle strength, and decreased endurance. The particular profile of positive and negative symptoms often corresponds to the specific insult to the nervous system.

Various medical conditions and external factors, including diabetes (i.e., diabetic neuropathy or DN), hypothyroidism, uremia, nutritional deficiencies, herpes zoster (shingles), alcoholism, stroke, HIV, multiple sclerosis, cancer and exposure to toxic substances, including chemotherapy (primarily chemotherapy with vincristine, cisplatin, zalcitabine, and paclitaxel) have been associated with neuropathic pain. Other acquired and inherited disorders, including Guillain-Barré syndrome (GBS), postherpetic neuralgia (PHN), Charcot-Marie-Tooth (CMT) disease, complex regional pain syndrome type 1 (CRPS-1), ischemic neuropathy, painful spasticities, and other nervous system disorders that have pain as an attendant sign and/or symptom may also be associated with neuropathic pain (Carter et al., Physical Medicine and Rehabilitation Clinics of North America, 12(2):447-59, 2001.

Models for neuropathic pain do not test pain response by a healthy nervous system. Rather, neuropathic pain models test the abnormal pain response resulting from damaged nerve tissue. One model produces neuropathic pain in test animals by surgically ligating spinal nerves (Kim and Chung et al., Pain, 50:355-363, 1992). The model provides a widely accepted model for peripheral neuropathic pain in humans. The model detects antihyperalgesic activity in rats suffering from neuropathic pain by employing a surgical procedure to form a spinal nerve ligature. The spinal nerve ligature produces a constriction injury that serves to model the perturbations associated with peripheral neuropathic injury in a mammal. Specifically, the phenomena of thermal hyperalgesia, cold allodynia, and tactile allodynia manifest themselves. Subsequent to the surgery to create the constriction injury, the rats are challenged with thermal and mechanical stimuli to determine the degree of sensitivity. Demonstration of an ability to decrease the abnormal pain sensitivity effected by the spinal nerve ligature is predictive of an agent's potential efficacious treatment of neuropathic pain.

Neuropathic pain is relatively resistant to NSAIDs and opioids, although they may be helpful in certain cases. Other major classes of medications useful for neuropathic pain include tricyclic antidepressants, anticonvulsants, and sodium channel blockers.

Fibromyalgia

Fibromyalgia is a syndrome which is a frequent cause of chronic, widespread pain and is estimated to affect 2-4% of the population. Fibromyalgia is characterized by a generalized heightened perception of sensory stimuli. Patients with fibromyalgia display abnormalities in pain perception in the form of both allodynia (pain with innocuous stimulation) and hyperalgesia (increased sensitivity to painful stimuli). Clinically, fibromyalgia is characterized by general aches or stiffness, primarily musculoskeletal in origin, involving three or more anatomical sites for at least three months and at least six typical and reproducible tender points. Other associated symptoms of fibromyalgia include fatigue, nonrestorative sleep and memory difficulties.

Fibromyalgia is likely to be caused by dysfunction of various components of the central nervous system (Yunus, J. Rheumatol., 19:846-850, 1993). Evidence has accumulated that aberrant function of the autonomic nervous system, and in particular the sympathetic nervous system, is responsible for the symptoms of fibromyalgia.

Abnormal findings in fibromyalgia patients strongly indicate a neuropathic pain syndrome, reminiscent of complex regional pain syndrome or postherpetic neuralgia. In addition, fibromyalgia seems to share similar characteristics with these neuropathic pain syndromes, including ineffective response to many analgesics (Staud, Pain Med., 2(3):208-15, 2001).

Some medications currently employed to treat fibromyalgia include, but are not limited to, analgesics, hypnotics, and immune suppressants. Though numerous agents have been used to treat fibromyalgia, no single pharmacological agent or combination of agents has been demonstrated to be effective.

Agents presently used to treat neuropathic pain or fibromyalgia are not always effective. Some may produce serious side effects. Some, such as opioid analgesics, may have serious addictive liability. There is a need for agents which are effective in treating pain, particularly neuropathic pain, and also fibromyalgia. In particular, there is a need for agents with few side effects and low liability for addiction, that are appropriate for long-term use in treatment and prevention of these disorders.

### SUMMARY OF THE INVENTION

According to the present invention, therapeutic methods are provided for treating pain or fibromyalgia. The method comprises administering to the individual an effective amount of a 1-[3-[4-(3-chlorophenyl)-1-piperazinyl]propyl]-5-methoxy-3,4-dihydro-2(1H)-quinolone, a prodrug thereof, or a pharmaceutically acceptable salt thereof. The preferred salt is the monomethanesulfonate. The monomethanesulfonate of 1-[3-[4-(3-chlorophenyl)-1-piperazinyl]propyl]-5-methoxy-3,4-dihydro-2(1H)-quinolone is also known as “OPC-14523”.

According to some embodiments of the pain treatment method of the invention, the pain is neuropathic pain. In other embodiments of the pain treatment method, the pain is nociceptive pain. In yet other embodiments of the pain treatment method, the pain is pain associated with fibromyalgia.

According to one embodiment, the neuropathic pain is associated with a metabolic insult other than diabetes. According to another embodiment, the neuropathic pain is associated with a physical or chemical injury, particularly a physical injury comprising nerve root compression.

According to another aspect of the invention, 1-[3-[4-(3-chlorophenyl)-1-piperazinyl]propyl]-5-methoxy-3,4-dihydro-2(1H)-quinolone, a prodrug thereof, or a pharmaceutically acceptable salt thereof, is used in the preparation of a medicament for treating or preventing pain or fibromyalgia.

As used in the specification and the appended claims, the singular forms “a,” “an” and “the” include plural referents unless the context clearly dictates otherwise.

The terms “effective amount” and “pharmaceutically effective amount” refer to a nontoxic but sufficient amount of an agent to provide the desired biological result. That result can be reduction and/or alleviation of the signs, symptoms, or causes of a disease, or any other desired alteration of a biological system. An appropriate effective amount in any individual case may be determined by one of ordinary skill in the art using routine experimentation.

As used herein, the terms “treat” and “treatment” are used interchangeably and are meant to indicate a postponement of development of a disorder and/or a reduction in the severity of symptoms that will or are expected to develop. The terms further include ameliorating existing symptoms, preventing additional symptoms, and ameliorating or preventing the underlying metabolic causes of symptoms.

By “pharmaceutically acceptable” and “pharmacologically acceptable” is meant a material which is not biologically or otherwise undesirable, i.e., the material may be administered to an individual without causing any undesirable biological effects or interacting in a deleterious manner with any of the components of the composition in which it is contained.

As used herein, “individual” (as in the subject of the treatment) means both mammals and non-mammals. Mammals include, for example, humans; non-human primates, e.g. apes and monkeys; cattle; horses; sheep; and goats. Non-mammals include, for example, fish and birds.

### DESCRIPTION OF THE FIGURES

FIG. 1 shows the results of acute administration of OPC-14523 or morphine on tactile stimulation in a model of neuropathic pain originally devised by Kim and Chung et al. (Pain, 50:355-363, 1992). Paw-withdrawal force values are the mean of data from eight rats for each compound. Paw-withdrawal force values at the zero time point are pre-test values. Drug doses indicated in parenthesis in the legend in this and all other figures are in mg/kg body weight. For this and all other figures, *=p<0.05; **=p<0.01; and ***=p<0.001.

FIG. 2 shows the results of acute administration of OPC-14523 or morphine on thermal stimulation in the same neuropathic pain model. Paw-withdrawal latency values are the mean of data from eight rats for each compound.

FIG. 3 shows the results of chronic administration of OPC-14523 or morphine on tactile stimulation in the neuropathic pain model. Paw-withdrawal force values are the mean of data from eight rats for each compound.

FIG. 4 shows the results of chronic administration of OPC-14523 or morphine on thermal stimulation in the neuropathic pain model. Paw-withdrawal latency values are the mean of data from eight rats for each compound.

FIG. 5 shows the results of acute administration of OPC-14523 or morphine on tactile stimulation in the neuropathic pain model from a second experiment. Paw-withdrawal force values are the mean of data from eight rats for each compound. Paw-withdrawal force values at the zero time point are pre-test values.

FIG. 6 shows the results of acute administration of OPC-14523 or morphine on cold stimulation in the neuropathic pain model with respect to the paw-withdrawal latency for the first withdrawal. Paw-withdrawal latency values are the mean of data from eight rats for each compound.

FIG. 7 shows the results of acute administration of OPC-14523 or morphine on cold stimulation in the neuropathic pain model with respect to the total number of paw withdrawals. Values of the total number of paw withdrawals are the mean of data from eight rats for each compound.

FIG. 8 shows the results of acute administration of OPC-14523 or morphine on cold stimulation in the neuropathic pain model with respect to the total duration of paw withdrawals. Total duration of paw withdrawal values are the mean of data from eight rats for each compound.

FIG. 9 shows the results of acute administration of (i) OPC-14523, (ii) a compound “A”, (iii) a compound “B” or (iv) gabapentin, on tactile stimulation in the neuropathic pain model measured using von Frey filaments. The log stimulus values are the mean of data from 10 rats for each compound administered. “N” is the mean threshold value on the day before ligature surgery. “L” is the mean threshold value four days after ligature surgery. Statistical significance was calculated by comparison to the corresponding value in the vehicle control group by two-way ANOVA followed by the Duncan test.

FIG. 10 shows the dose dependency results of acute administration of OPC-14523 or gabapentin on tactile stimulation in the Chung neuropathic pain model measured using von Frey filaments. The log stimulus values are the mean of data from eight rats for each compound/dose administered. Statistical significance of the difference in threshold was calculated by comparison to the corresponding value in the vehicle control group by two-way ANOVA followed by the Duncan test.

### DETAILED DESCRIPTION OF THE INVENTION

According to the present invention, 1-[3-[4-(3-chlorophenyl)-1-piperazinyl]propyl]-5-methoxy-3,4-dihydro-2(1H)-quinolone, a prodrug thereof, or a pharmaceutically acceptable salt thereof, is administered to reduce pain. The compound may also be employed in the prevention or treatment of fibromyalgia.

1-[3-[4-(3-Chlorophenyl)-1-piperazinyl]propyl]-5-methoxy-3,4-dihydro-2(1H)-quinolone has the formula 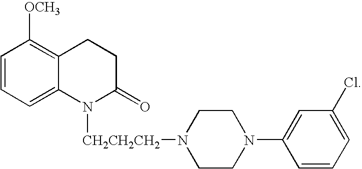
The compound and its pharmaceutically acceptable salts may be prepared according to the synthetic methods described in U.S. Pat. Nos. 5,556,857 and 5,656,633 and Oshiro et al., J. Med. Chem. 43:177-189 (2000), the entire disclosures of which are incorporated herein by reference. The monomethanesulfonate is compound 34c of Oshiro et al.

The compound 1-[3-[4-(3-chlorophenyl)-1-piperazinyl]propyl]-5-methoxy-3,4-dihydro-2(1H)-quinolone, prodrug thereof, or pharmaceutically acceptable salt thereof, may be administered to any subject for treating pain. The pain may be nociceptive. The nociceptive pain may be somatic or visceral pain.

The pain treated according to the present invention may also be neuropathic pain. Neuropathic pain occurs when the peripheral and/or central nervous systems are sensitized following an injury to the peripheral system. This initial injury can occur from traumatic physical injury as well as metabolic insult. By “metabolic insult” is meant an injury to or attack on either (1) the physical or chemical processes by which living organized substance is produced and maintained (anabolism), or (2) the transformation by which energy is made available for the uses of the organism (catabolism) (cf. Dorland's Illustrated Medical Dictionary, 29th ed., Philadelphia, Saunders, 2000). A metabolic insult can be caused by a systematic disease. Systemic diseases include, for example, herpes zoster, AIDS/HIV, syphilis and various other autoimmune diseases. Another systemic disease is diabetes. Neuropathic pain may be induced by diabetic conditions (diabetic neuropathy). Neuropathy of primary afferent axons in long nerves is found in diabetic patients.

Other metabolic insults which can give rise to neuropathic pain treatable according to the present invention include chemotherapy-related neuropathies, such as neuropathies caused by chemotherapy with vincristine, cisplatin, zalcitabine, or paclitaxel; hypothyroidism; uremia; nutritional deficiencies; alcoholism; stroke; multiple sclerosis; cancer; and exposure to toxic substances. Other acquired and inherited disorders, including Guillain-Barré syndrome (GBS); neuralgias, such as postherpetic neuralgia (PHN); Charcot-Marie-Tooth (CMT) disease; complex regional pain syndrome type 1 (CRPS-1); ischemic neuropathy; painful spasticities; and other nervous system disorders that have pain as an attendant sign and/or symptom may also be associated with neuropathic pain.

According to one embodiment of the invention, the treated neuropathic pain is neuropathic pain arising from physical or chemical injury. A “chemical injury” includes, for example, injury caused by chemotherapy or toxic chemical exposure. According to another embodiment of the invention, the treated neuropathic pain is neuropathic pain arising from a metabolic insult, such as an insult resulting from a systemic disease. According to one sub-embodiment, the treated neuropathic pain is neuropathic pain arising from a metabolic insult other than diabetes.

According to one embodiment of the invention, the neuropathic pain is pain associated with an injury or condition comprising nerve root compression. The patient typically feels pain at the site where sensitivity is picked up by the root, and not at the compressed site. Failed back pain and sciatic pain in particular, is an example of nerve root pain. Other manifestations of neuropathic pain from injury include, for example, phantom limb pain, trauma that damages tissue and nerves, and burns (that burn skin as well as nerve endings).

The compounds used in the practice of methods of the present invention may take the form of pharmaceutically acceptable salts. The term “salts” embraces salts commonly used to form alkali metal salts and to form addition salts of free acids or free bases. The term “pharmaceutically acceptable salt” refers to salts that possess toxicity profiles within a range so as to have utility in pharmaceutical applications. Suitable pharmaceutically acceptable acid addition salts may be prepared from an inorganic acid or from an organic acid. Examples of such inorganic acids are hydrochloric, hydrobromic, hydroiodic, nitric, carbonic, sulfuric and phosphoric acid. Appropriate organic acids may be selected from aliphatic, cycloaliphatic, aromatic, araliphatic, heterocyclic, carboxylic and sulfonic classes of organic acids, example of which are formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, salicyclic, 4-hydroxybenzoic, phenylacetic, mandelic, embonic (pamoic), methanesulfonic, ethanesulfonic, benzenesulfonic, pantothenic, 2-hydroxyethanesulfonic, toluenesulfonic, sulfanilic, cyclohexylaminosulfonic, stearic, algenic, beta-hydroxybutyric, galactaric, and galacturonic acid.

The compound may also take the form of a prodrug of 1-[3-[4-(3-chlorophenyl)-1-piperazinyl]propyl]-5-methoxy-3,4-dihydro-2(1H)-quinolone. Prodrugs according to this invention are inactive derivatives of 1-[3-[4-(3-chlorophenyl)-1-piperazinyl]propyl]-5-methoxy-3,4-dihydro-2(1H)-quinolone that are metabolized in vivo into the active agent in the body. Prodrugs useful according to this invention are those that have therapeutic value substantially the same or better than 1-[3-[4-(3-chlorophenyl)-1-piperazinyl]propyl]-5-methoxy-3,4-dihydro-2(1H)-quinolone in treating pain or fibromyalgia. For example, a prodrug useful according to this invention can improve the penetration of the drug across biological membranes leading to improved drug absorption; prolong duration of the action of the drug, e.g., slow release of the parent drug from the prodrug and/or decrease first-pass metabolism of the drug; target the drug action; improve aqueous solubility and stability of the drug (e.g., intravenous preparations); improve topical drug delivery (e.g., dermal drug delivery); improve the chemical and/or enzymatic stability of drugs; or decrease side effects due to the drug. Methods for making prodrugs are know in the art (e.g., Balant, Eur. J. Drug Metab. Pharmacokinet. 15:143-153, 1990; and Bundgaard, Drugs of the Future 16:443-458, 1991; incorporated herein by reference).

For treating pain and for treating fibromyalgia, the specific dose of compound according to the invention to obtain therapeutic benefit will, of course, be determined by the particular circumstances of the individual patient, including, the size, weight, age, and sex of the patient, the nature and extent of the condition being treated, and recommendations of the treating physician. Also determinative will be the stage of the disease and the route of administration. Thus, the effective amount for a given situation can be determined by routine experimentation. Generally a therapeutic amount will be in the range of about 0.01 mg/kg to about 40 mg/kg body weight, more preferably about 0.1 mg/kg to about 10 mg/kg, in at least one dose. In larger mammals the indicated dosage can be from about 1 mg to 300 mg, one or more times per day, more preferably in the range of about 10 mg to 200 mg. The subject may be administered as many doses as is required to reduce and/or to alleviate the signs, symptoms, or causes of the disorder in question. For example, a daily dosage of from about 1 to 1500 mg/day may be utilized. Preferably, a daily dosage of from about 5 mg/day to 500 mg/day may be utilized. More preferably, a daily dosage of from about 5 mg/day to 100 mg/day may be utilized. Higher or lower doses are also contemplated.

The methods of the present invention may comprise administering the compound in the form of a pharmaceutical composition, in combination with a pharmaceutically acceptable carrier. The active ingredient in such formulations may comprise from 0.1 to 99.99 weight percent. By “pharmaceutically acceptable carrier” is meant any carrier, diluent, or excipient that is compatible with the other ingredients of the formulation and not deleterious to the recipient.

The compound may be administered for therapeutic effect by any route, for example enteral (oral, rectal, intranasal, etc.) and parenteral administration. Parenteral administration includes, for example, intravenous, intramuscular, intraarterial, intraperitoneal, intravaginal, intravesical (for example, into the bladder), intradermal, topical, and subcutaneous administration. Also contemplated within the scope of the invention is the instillation of drug in the body of the patient in a controlled formulation, with systemic or local release (such as, for example, in the gastrointestinal tract) of the drug to occur at a later time.

The active agent is preferably administered with a pharmaceutically acceptable carrier selected on the basis of the chosen route of administration and standard pharmaceutical practice. The active agent may be formulated into dosage forms according to standard practices in the field of pharmaceutical preparations. (See Gennaro, editor, Remington's Pharmaceutical Sciences, 18th ed., Easton, Pa., Mack Publishing Co., 1990). Suitable dosage forms may comprise, for example, tablets, capsules, solutions, parenteral solutions, troches, suppositories, or suspensions.

For parenteral administration, the active agent may be mixed with a suitable carrier or diluent such as water, an oil (particularly a vegetable oil), ethanol, saline solution, aqueous dextrose (glucose) and related sugar solutions, glycerol, or a glycol such as propylene glycol or polyethylene glycol. Solutions for parenteral administration preferably contain a water-soluble salt of the active agent. Stabilizing agents, antioxidizing agents, and preservatives may also be added. Suitable antioxidizing agents include sulfite, ascorbic acid, citric acid and its salts, and sodium EDTA. Suitable preservatives include benzalkonium chloride, methyl- or propyl-paraben, and chlorobutanol. The composition for parenteral administration may take the form of an aqueous or nonaqueous solution, dispersion, suspension, or emulsion.

For oral administration, the active agent may be combined with one or more solid inactive ingredients for the preparation of tablets, capsules, pills, powders, granules, or other suitable oral dosage forms. For example, the active agent may be combined with at least one excipient such as fillers, binders, humectants, disintegrating agents, solution retarders, absorption accelerators, wetting agents, absorbents, or lubricating agents. According to one tablet embodiment, the active agent may be combined with carboxymethylcellulose calcium, magnesium stearate, mannitol, and starch, and then formed into tablets by conventional tableting methods.

The compositions of the present invention can also be formulated so as to provide slow or controlled release of the active ingredient therein. In general, a controlled release preparation is a composition capable of releasing the active ingredient at the rate required to maintain constant pharmacological activity for a desirable period of time. Such dosage forms can provide a supply of a drug to the body during a predetermined period of time and thus maintain drug levels in the therapeutic range for longer periods of time than other non-controlled formulations.

The controlled release of the active ingredient may be stimulated by various inducers, for example, pH, temperature, enzymes, water, or other physiological conditions or compounds. Various mechanisms of drug release exist. For example, in one embodiment, the controlled-release component can swell and form porous openings large enough to release the active ingredient after administration to a patient. The term “controlled-release component” in the context of the present invention is defined herein as a compound or compounds, such as polymers, polymer matrices, gels, permeable membranes, liposomes and/or microspheres, that facilitate the controlled-release of the active ingredient (for example, the compound or a pharmaceutically acceptable salt thereof) in the pharmaceutical composition. In another embodiment, the controlled-release component is biodegradable, induced by exposure to the aqueous environment, pH, temperature, or enzymes in the body. In another embodiment, sol-gels can be used, wherein the active ingredient is incorporated into a sol-gel matrix that is a solid at room temperature. This matrix is implanted into a patient, preferably a mammal, having a body temperature high enough to induce gel formation of the sol-gel matrix, thereby releasing the active ingredient into the patient.

The compound is administered according to the present invention to patients suffering from conditions that manifest the symptoms of pain or fibromyalgia.

The practice of the invention is illustrated by the following non-limiting examples.

Examples 2 and 3 on the one hand, and Examples 4 and 5 on the other hand, utilize slightly different pain models. Two nerves, L5 and L6, were ligated in the spinal ligation surgery of Examples 2 and 3. Only L5 was ligated in Examples 4 and 5. The rats in Examples 2 and 3 recovered for at least two weeks between surgery and data collection. Rats in Example 2 were used again in Example 3. In Examples 4 and 5, data were collected on the fourth day post-surgery. Tactile allodynia was measured using an electronic von Frey probe in Examples 2 and 3. The probe uses a single stimulation of increasing pressure. Von Frey filaments were used in Examples 4 and 5. Von Frey filaments comprise sequential applications of filaments of different stiffness.

### EXAMPLES

### Example 1

### Preparation of Immediate Release Tablet

Tablets were prepared containing 5 mg active ingredient (OPC-14523). The tablets contained, in addition to the active ingredient, lactose monohydrate (101.0 mg), corn starch (20.0 mg), microcrystalline cellulose (20.0 mg), low-substituted hydroxypropyl cellulose (20.0 mg), hydroxypropyl cellulose (4.0 mg), hydrogenated castor oil (4.0 mg), hydroxypropyl methyl cellulose (4.8 mg), titanium dioxide (0.6 mg), talc (0.6 mg) and purified water (145.0 mg).

### Example 2

### Evaluation of Acute and Chronic Administration of OPC-14523 in a Model of Neuropathic Pain Using Tactile Stimulation and Thermal Stimulation

The Chung neuropathic pain model (Kim and Chung, Pain, 50: 355-363, 1992), employs spinal nerve ligation (SNL) in a rat. Tight ligature of spinal nerves in a rat is associated with hyperalgesia, allodynia and spontaneous pain. Antihyperalgesics reduce these chronic signs of pain hypersensitivity. Thus, the Chung model permits testing of the antihyperalgesic activity of compounds in rats with neuropathic pain. The Chung model constitutes a model for peripheral neuropathic pain in humans, and was employed as follows.

### I. Materials and Methods

A. Animals

Male Rj: Wistar (Han) rats, weighing 264-369 g at the beginning of the experiment, were obtained from Elevage Janvier, 53940 Le Genest-Saint-Isle, France. The rats were acclimatized to laboratory conditions for at least 5 days before the surgery phase of the experiment. The rats were housed in groups of five in macrolon cages (41×25×14 cm) on wood litter (Litalabo—SPPS, 95100 Argenteuil, France) with free access to food (code 113—SAFE, 91360 Epinay-sur-Orge, France) and water until tested. The animal houses were maintained under artificial lighting (12 hours) between 7:00 AM and 7:00 PM in a controlled ambient temperature of 21±3° C., and with relative humidity maintained at 40-70%.

B. Surgery

Rats (264-369 g) were anesthetized with sodium pentobarbital (50 mg/kg i.p.), and an incision at the L4-S2 level was made to expose the left L5 and L6 spinal nerves. A ligature was tied tightly around each nerve. The wound was then sutured. The rats received an i.m. injection of 50,000 IU penicillin G and were allowed to recover. At least 2 weeks after the surgery, when the chronic state was fully installed, rats were submitted consecutively to tactile and thermal stimulation of both the non-lesioned and the lesioned hindpaws.

C. Compound Preparation and Dosing

Doses of OPC-14523 were prepared in stock solutions (weight per volume, W/V), then in dilutions (volume per volume, V/V), as appropriate. The compound was dispersed in 0.5% tragacanth in distilled water. Morphine was dissolved in physiological saline as a reference standard for treatment of pain. The solutions for the test and reference substances were prepared fresh on the day of the experiment.

The vehicle, OPC-14523, and reference (morphine) compound were each administered in a volume of 5 ml/kg body weight. The doses of OPC-14523 were expressed in mg of substance per kg body weight and administered orally (p.o.). The vehicle control was also administered orally. The dose of morphine was expressed in mg of hydrochloride salt per kg body weight and administered i.p.

Each animal was tested at 30 minutes, 1 hour, 2 hours and 4 hours after the administration of a substance. Testing occurred in the morning.

To evaluate chronic drug administration and hypersensitivity induction, animals received a second substance treatment in the afternoon, after acute testing. The animals then received twice-daily substance treatment for an additional 9 days. To test for induction of hypersensitivity, the animals were tested, on the ninth day, 30 minutes before the first substance administration in the morning. The effect of chronic administration was tested on the tenth day 30 minutes, 1 hour, 2 hours and 4 hours after the administration of the substance in the morning.

D. Behavioral Testing

1. Tactile Stimulation

For tactile stimulation, the animal was placed under an inverted Plexiglas® box (17×11×14 cm) on a grid floor. The tip of an electronic Von Frey probe (Bioseb, Model 1610, Chaville, France) was applied with increasing pressure to the non-lesioned hindpaw or the lesioned hindpaw. The force required to induce paw withdrawal was automatically recorded. This procedure was carried out three times for each paw, and the mean force per paw was calculated to provide basic scores per animal.

2. Thermal Stimulation

An apparatus (Ugo Basile, Product reference: 7371, Comerio, Italy) consisted of six individual Plexiglas® boxes (17×11×14 cm) placed upon an elevated glass floor. A rat was placed in the box and left free to habituate for 10 minutes. Then, a mobile infrared radiant source was focused under the non-lesioned and lesioned hindpaws. The paw-withdrawal latencies were automatically recorded. Paw withdrawal interrupts the reflected radiation and switches off the counter and the light source. In order to prevent tissue damage, the test was terminated after 45 seconds if no reaction was noted.

Prior to receiving drug treatment, all animals were submitted to tactile stimulation and assigned to treatment groups, matched on the basis of their pain response. Each treatment group consisted of eight rats. The testing was performed blind. Animals were maintained in the same treatment groups for evaluation of hypersensitivity induction and chronic administration.

3. Data Analysis

Data were analyzed using commercial software (Microsoft Excel®, Microsoft Corp, Redmond, Wash. and GB Stat® version 6.5, Dynamic Microsystems, Inc., Silver Spring, Md.). All differences were considered statistically relevant when the null hypothesis could be rejected at the risk α≦0.05.

For vehicle treatment, values for the lesioned paw were compared with the non-lesioned paw using the paired Student's t test. The lesioned paw data were always statistically significant compared to the non-lesioned paw data. For OPC-14523 and morphine treatments, paw data were compared with the vehicle paw data using non-paired Student's t tests.

### II. Results

A. Acute Administration

1. Tactile Stimulation

Spinal nerve ligature produced a clear decrease in the force that would induce paw withdrawal after tactile stimulation of the lesioned paw of vehicle controls, as compared with the non-lesioned paw (−84%, −84%, −85% and −81% at 30 minutes, 1 hour, 2 hours, and 4 hours, respectively: p<0.001). The observed decrease in the force inducing paw withdrawal was sustained, in the absence of treatment, throughout the experiments which employed these rats.

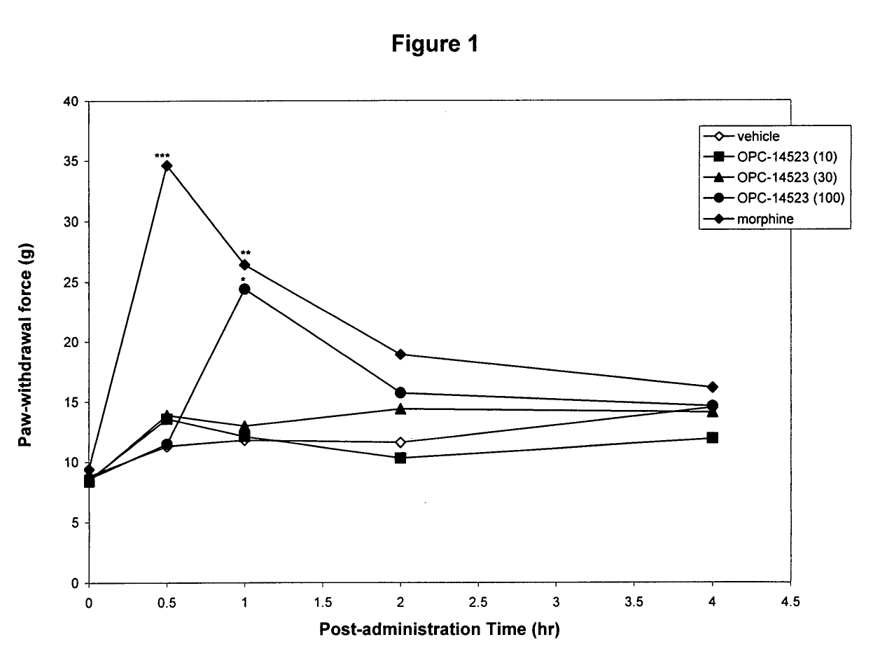

The reference substance, morphine (16 mg/kg, i.p.), markedly increased the force inducing paw withdrawal in the lesioned paw at both 30 minutes and 1 hour after administration (increase of +206% and +124%, respectively; FIG. 1). At this dose, morphine also tended to increase the force inducing paw withdrawal at 2 hours. It had no effects in the non-lesioned paw. OPC-14523 at 100 mg/kg p.o. clearly increased the force inducing paw withdrawal in the lesioned paw measured 1 hour after substance administration, compared with the vehicle. The increase was 107%. At this dose, OPC-14523 also tended to increase the force inducing paw withdrawal at 2 hours. OPC-14523 slightly decreased the force inducing paw withdrawal in the non-lesioned paw at 100 mg/kg at 4 hours (−27%, p<0.01). It had no significant effects at other doses or time points.

2. Thermal Stimulation

Spinal nerve ligature clearly induced a clear decrease in the paw-withdrawal latency after thermal stimulation in the lesioned paw of the vehicle, compared to the non-lesioned paw (−51%, p<0.001; −51%, p<0.05; −60%, p<0.001; and −82%, p<0.001 at 30 minutes, 1 hour, 2 hours, and 4 hours, respectively). This observed decrease in paw-withdrawal latency was sustained, in the absence of treatment, throughout the experiments that employed these rats.

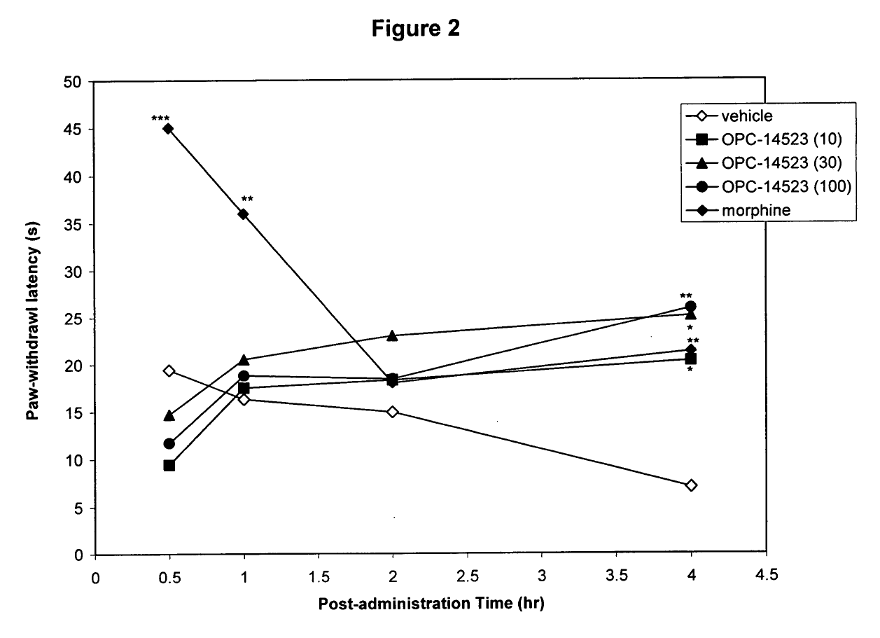

Morphine (16 mg/kg, i.p.; positive control) tended to increase the paw-withdrawal latency in the lesioned paw (FIG. 2). This tendency was statistically significant at 30 minutes, 1 hour and 4 hours after administration, compared to the vehicle (+132%, +121% and +204%, respectively). It tended to increase the paw-withdrawal latency in the non-lesioned paw. At 30 minutes after administration, the increase was statistically significant (+15%, p<0.05). OPC-14523 markedly and dose-dependently increased the paw-withdrawal latency in the lesioned paw, 4 hours after administration (+190%, 259% and 270% at 10, 30 and 100 mg/kg p.o., respectively; FIG. 2). At 10 mg/kg p.o., OPC-14523 tended to decrease latency at 30 minutes (−57%, NS). It tended to slightly decrease paw-withdrawal latency in the non-lesioned paw (statistically significant only at 100 mg/kg p.o., −27%, p<0.05).

Overall, the data indicate that acute administration of OPC-14523 is generally effective against both tactile allodynia (100 mg/kg p.o.) and thermal hyperalgesia (10-100 mg/kg p.o.).

B. Chronic Administration

1. Tactile Stimulation

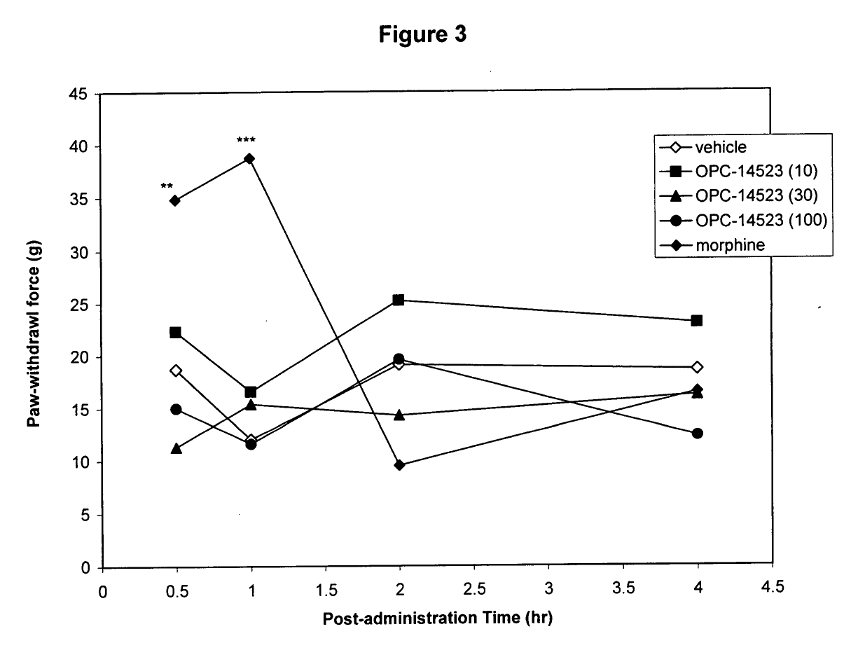

After 10 days, OPC-14523 did not have clear effects on the force inducing paw withdrawal in either the lesioned (FIG. 3) or the non-lesioned paw. Morphine markedly increased the force inducing paw withdrawal in the lesioned paw, measured 30 minutes and 1 hour after administration (+86%, p<0.01 and +223%, p<0.001, respectively). However, morphine tended to have the opposite effect at 2 hours after administration (−50%, p=0.09). Morphine had weak, variable effects in the non-lesioned paw.

2. Thermal Stimulation

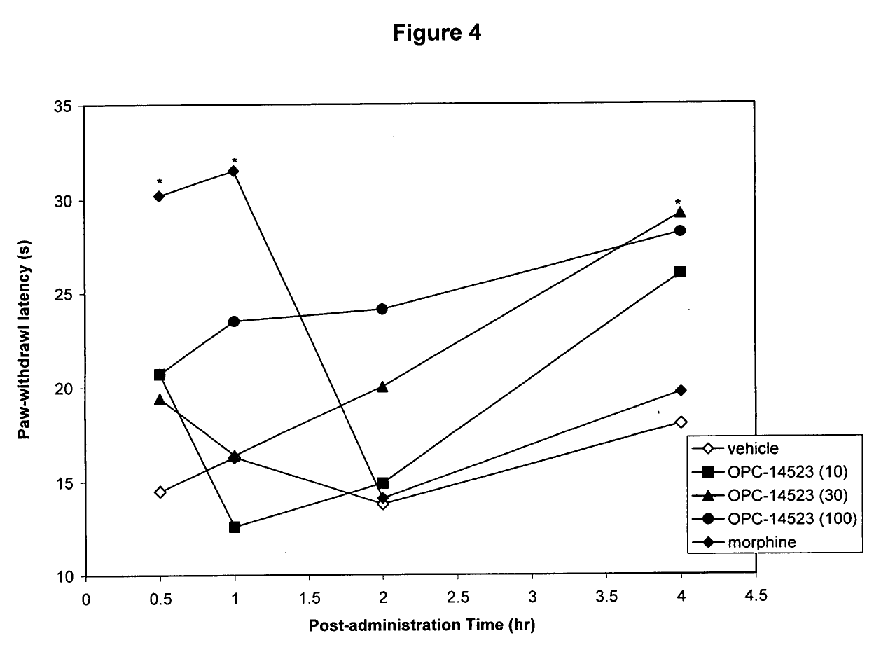

Morphine (16 mg/kg i.p.) tended to increase the paw-withdrawal latency in the lesioned paw (FIG. 4). This effect was statistically significant at 30 minutes and 1 hour after administration (+108%, p<0.05, and 93%, p<0.05, respectively). It tended to decrease the paw-withdrawal latency in the non-lesioned paw. This effect was statistically significant at 2 hours and 4 hours after administration (−24%, p<0.05, and −23%, p<0.05, respectively). OPC-14523 tended to increase the paw-withdrawal latency after thermal stimulation in the lesioned paw, though this effect was statistically significant only at 30 mg/kg at 4 hours after administration (+62%, p<0.05; FIG. 4). It had no effect on the non-lesioned paw.

Overall, these data suggest that OPC-14523 has activity against hyperalgesia after chronic administration.

C. Hypersensitivity

Hypersensitivity in the rats was measured by both tactile and thermal stimulation after 8 days of twice daily compound administration. In rats repeatedly treated with OPC-14523 at three different doses or with morphine, no significant effects were seen on the force inducing paw withdrawal in the lesioned or non-lesioned paw, compared with the vehicle control (Table 1).

TABLE 1

| | Force Inducing Paw | Force Inducing Paw |
|---|---|---|
| Treatment | Withdrawal (g) | Withdrawal (g) |
| (mg/kg) | Lesioned Paw | Non-lesioned Paw |
| Vehicle | 12.9 (p < 0.0001)† | 76.9 |
| OPC-14523 (10) | 17.6 (NS) | 72.9 (NS) |
| OPC-14523 (30) | 15.1 (NS) | 83.5 (NS) |
| OPC-14523 (100) | 16.2 (NS) | 78.1 (NS) |
| Morphine | 15.1 (NS) | 73.7 (NS) |

†Compared to vehicle non-lesioned paw
NS—Not significant compared to corresponding vehicle paw

As shown in Table 2, in rats repeatedly treated with OPC-14523 at 10 mg/kg p.o., a decrease in paw-withdrawal latency was observed in the lesioned paw (−67%, p<0.01). No effects were seen on the paw-withdrawal latency in the lesioned paw at other test compound doses, or in the non-lesioned paw at any dose, compared to the vehicle control.

TABLE 2

| | | Paw Withdrawal |
|---|---|---|
| | Paw Withdrawal | Latency (s) |
| | Latency (s) | Non-lesioned |
| Treatment | Lesioned Paw | Paw |
| Vehicle | 13.8 (p < 0.001)† | 40.8 |
| OPC-14523 (10) | 4.5 (p < 0.01) | 43.7 (NS) |
| OPC-14523 (30) | 19.9 (NS) | 37 (NS) |
| OPC-14523 (100) | 22.3 (NS) | 39.1 (NS) |
| Morphine | 11.7 (NS) | 34.5 (NS) |

†Compared to vehicle non-lesioned paw
NS—Not significant compared to corresponding vehicle paw

Overall, these data indicate that repeated administration of OPC-14523 does not induce hypersensitivity.

### Example 3

### Evaluation of Acute Administration of OPC-14523 in a Model of Neuropathic Pain Using Tactile Stimulation and Cold Stimulation

### I. Materials and Methods

The rats used in this experiment were the same rats used in Example 2. The materials and methods for this experiment were identical to those for Example 2 with the following exceptions: there was a 1-week washout between the two experiments, and the rats weighed 290-403 g at the start of this experiment. Prior to receiving drug treatment, all animals were submitted to tactile stimulation and assigned to treatment groups matched on the basis of their pain response. No account was taken of the treatment administered during the first experiment in assigning animals to treatment groups for this experiment.

All of the substances were administered orally. Morphine, dissolved in distilled water, was administered at 128 mg/kg dose p.o. Rats were tested 30 minutes, 1 hour, 2 hours, and 4 hours after administration of the substances.

Behavioral testing was carried out by measuring cold allodynia using an apparatus consisting of a cold plate maintained at 4±1° C. Rats were individually placed on the cold plate for a period of 5 minutes. The latency to the first paw withdrawal response and the number and total duration of withdrawal responses in 5 minutes were recorded for both the lesioned and the non-lesioned hindpaws.

### II. Results

A. Tactile Stimulation

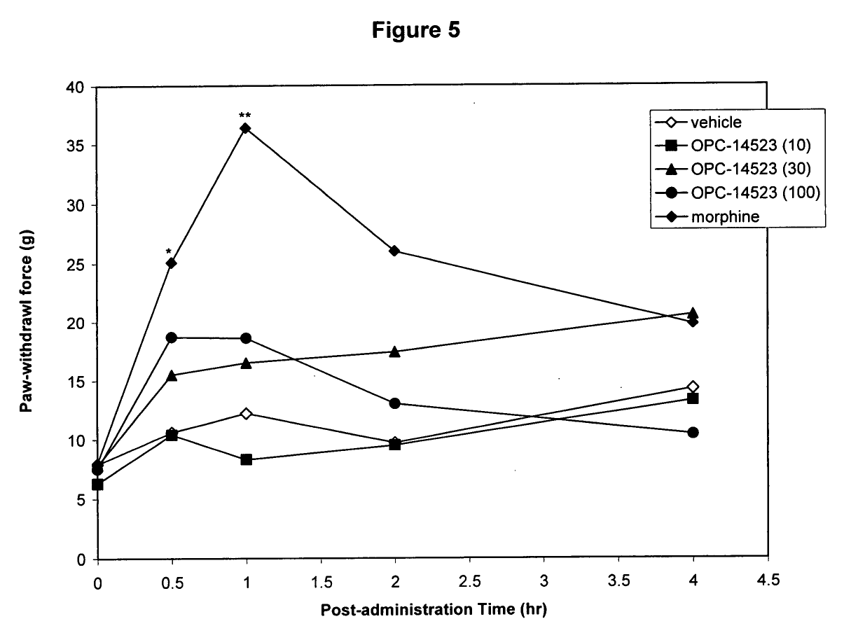

As shown in FIG. 5, morphine (128 mg/kg p.o.) markedly increased the force inducing paw withdrawal in the lesioned paw at 30 minutes and 1 hour after administration, as compared to the vehicle control (+137%, p<0.05 and +198%, p<0.01). Similarly, there was a tendency towards increase at 2 hours after administration (+168%, p=0.05). Morphine had no effects in the non-lesioned paw. OPC-14523 did not significantly affect the force inducing paw withdrawal in the lesioned paw (FIG. 5). A tendency towards an increase, however, was observed at 30 mg/kg and 100 mg/kg at 30 minutes after administration (+46%, p=0.10 and +76%, p=0.11, respectively), and at 30 mg/kg 2 hours after administration (+79%, p=0.11). OPC-14523 had no clear effects on the force inducing paw withdrawal in the non-lesioned paw.

B. Cold Allodynia

In the vehicle controls, as expected, the spinal nerve ligature resulted in the appearance of paw-withdrawal responses when a cold stimulus was applied to the lesioned paw, but not when the cold stimulus was applied to the non-lesioned paw. No withdrawal responses were recorded in the non-lesioned paws.

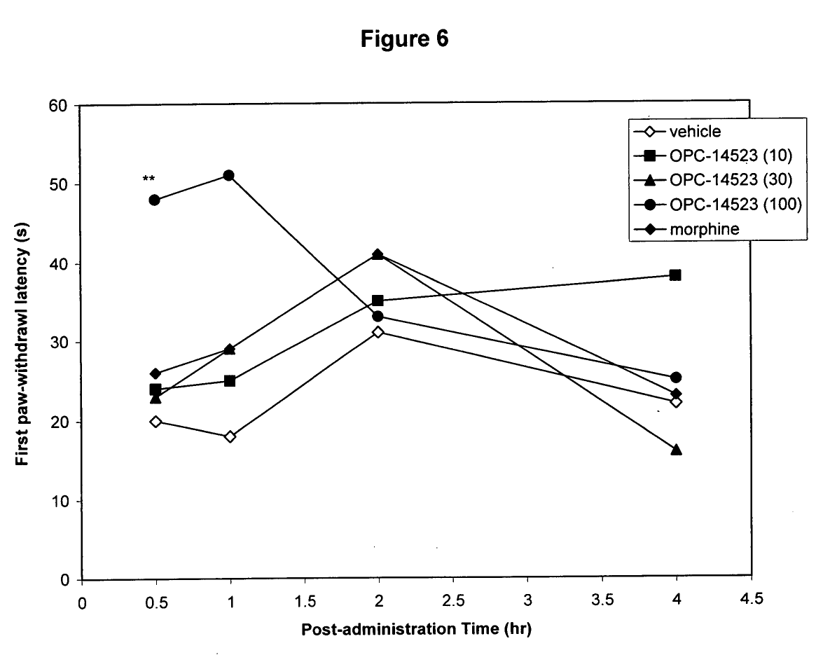

Morphine tended to increase paw-withdrawal latency of the first withdrawal, although not statistically significantly (FIG. 6). As shown in FIG. 6, the total number of paw withdrawal responses was reduced at all time points (−61%, p<0.001; −65%, p<0.05; −51%, p<0.01; and −56%, p<0.01 at 30 minutes, 1 hour, 2 hours and 4 hours, respectively).

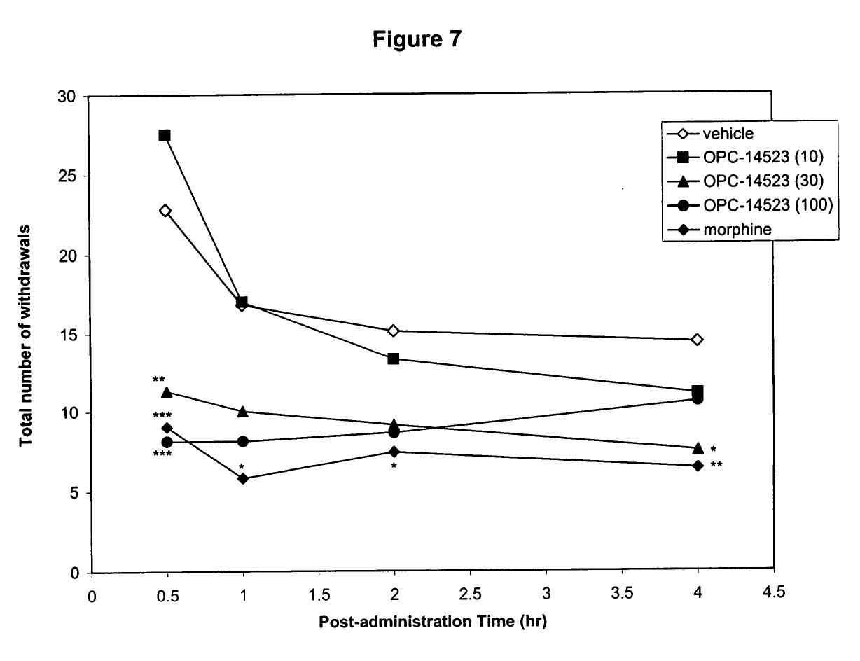

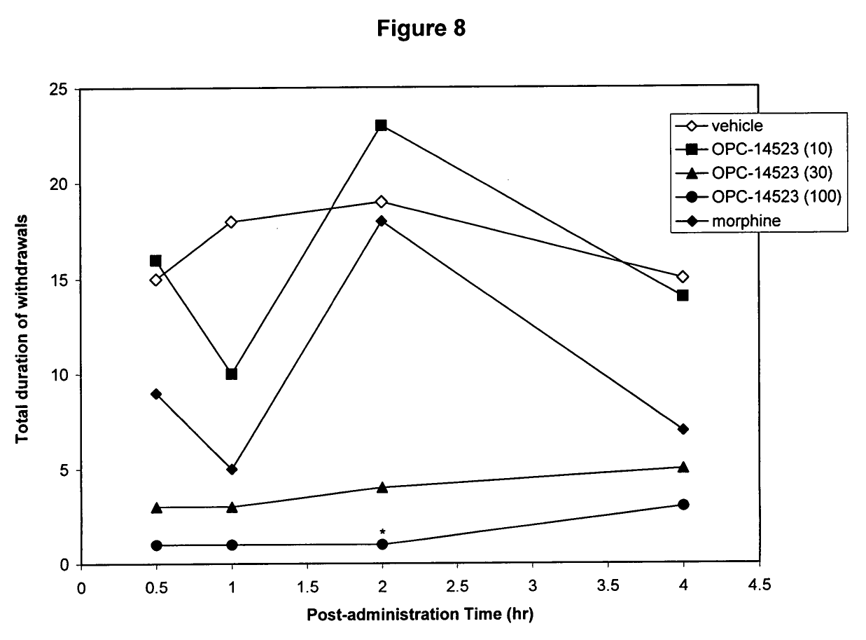

As shown in FIG. 6, at 100 mg/kg, OPC-14523 clearly increased the latency to paw withdrawal in the lesioned paw at 30 minutes (+140%, p<0.01). OPC-14523 also tended to increase latency at 1 hour (+183%, p=0.24). OPC-14523 dose-dependently decreased the number of withdrawal responses in the lesioned paw at 30 minutes (FIG. 7). Statistically significant decreases were observed at 30 mg/kg and 100 mg/kg p.o. at 30 minutes (50%, p<0.01 and 64%, p<0.05, respectively) and at 4 hours at 30 mg/kg (−48%, p<0.05), with a similar tendency at 1 and 2 hours. OPC-14523 also dose-dependently reduced the total duration of withdrawal responses, reaching statistical significance at 100 mg/kg p.o. at 2 hours (−95%, p<0.05; FIG. 8). Similar tendencies were observed at 30 mg/kg and 100 mg/kg, from 30 minutes to 2 hours.

Overall, these data indicate that OPC-14523 tended to increase the force inducing paw withdrawal after tactile stimulation. Furthermore, the data indicate that compound increased the paw-withdrawal latency after cold allodynia. OPC-14523 dose-dependently reduced both the number of paw withdrawals and the total duration of withdrawal responses after cold allodynia. These results, therefore, support the finding that OPC-14523 has efficacy in relieving neuropathic pain.

### Example 4

### Evaluation of Acute Administration of OPC-14523 in a Model of Neuropathic Pain Using Tactile Stimulation and Von Frey Filaments

### I. Materials and Methods

A. Animals

A total of 18 Sprague-Dawley (CD sub-strain) young adult (weight: 150-175 g) male rats were purchased from Harlan Sprague Dawley Company (Indianapolis, Ind.), Alabama colony. The rats were kept three per cage and had free access to food and water. Animals were kept in a room with a reversed light-dark cycle (light: 8:00 PM-8:00 AM) for 1 week before experimental manipulations. Ten rats were allocated to the present study. Eight rats were allocated to the study of Example 5, below.

B. Surgery

The spinal nerve ligation (SNL) model of neuropathic pain was produced in all rats as described by Kim and Chung (Pain, 50, 355-363, 1992). Briefly, under gaseous anesthesia using halothane (3% for induction and 1.5% for maintenance) in oxygen, the left L5 spinal nerve was exposed by removing the paraspinal muscles and the transverse process of the L6 vertebra. The very proximal portion of the L5 spinal nerve was tightly ligated with 6-0 silk suture. The wounds were closed and anesthesia was discontinued. Rats were kept on a heated blanket until they had completely recovered from anesthesia.

C. Compound Preparation and Dosing

OPC-14523 was dispersed in 0.5% tragacanth in distilled water at 20 mg/ml. It was administered as a oral injection in a dose of 100 mg/kg body weight. Gabapentin was prepared at 10 mg/ml in saline and was administered at 50 mg/kg body weight i.p. Gabapentin is a drug that was originally developed as an anticonvulsant but is now widely used as an analgesic for neuropathic pain (Abdi et al., Anesthesia & Analgesia 87:1360-1366 (1998)). The administered dose is the highest dose of systemic gabapentin that does not have obvious side effects.

D. Experimental Design:

Ten rats were utilized in a Latin-square-design study comparing the activity of OPC-14523 and other compounds against neuropathic pain. Behavioral tests were conducted twice (1 day and 1 hour) before, as well as 1 and 4 days after, the neuropathic surgery. On the fourth postoperative (PO) day, all rats showed similar levels of allodynic behaviors (lowering of the mechanical threshold for foot withdrawals). The ten rats were randomly divided into five groups of two rats each. Immediately after behavioral testing on the fourth PO day, rats in groups 1-4 received an i.p. injection of vehicle (0.2% hydroxypropylmethylcellulose [HPMC] in saline), compound A, compound B, or gabapentin, respectively. (Compounds A and B are test compounds unrelated to either gabapentin or OPC-14523.) Group 5 received an oral injection of OPC-14523. Behavioral tests were conducted 0.5, 1, 2, 4, and 6 hours after each injection. Then, all animals rested for 1 day (or until Monday on the weekend). On the sixth PO day, each group received one of the five compounds above that they had not received before.

This procedure was repeated five times. The overall experiment took about 2 weeks (injections on Monday, Wednesday, Friday, and the ensuing following Monday, and Wednesday, followed by behavioral testing and 1 day of rest after each injection). The result was that all ten animals received each of the indicated five compounds in random order and then had mechanical thresholds determined at the indicated times.

E. Behavioral Testing Tactile Stimulation

Behavioral tests were conducted in a non-blind fashion. The foot withdrawal threshold to mechanical stimuli applied to the paw (mechanical threshold) was measured, as an indicator of mechanical sensitivity of the affected paw. The foot withdrawal threshold was measured using von Frey filaments. It is generally thought that von Frey filaments are more sensitive than electronic von Frey probes for measuring tactile stimulation. The mechanical thresholds were measured by using the “up-and-down” method (Chaplan et al., J. Neurosci. Methods 53(1):55-63, 1994), following the procedures described in previous studies (Park et al., Pain 87(2):171-179, 2000; Xie et al., J. Pain 2(5):270-278, 2001; Baik et al., J. Pain 4:212-221, 2003). In brief, rats were placed in a transparent plastic box on a metal wire mesh floor. To determine the threshold stiffness required for 50% paw withdrawal, a series of eight von Frey filaments with approximately equal logarithmic incremental (0.22) von Frey values (3.65, 3.87, 4.10, 4.31, 4.52, 4.74, 4.92, and 5.16) were used. Von Frey (VF) values are logarithmically related to gram (g) values (VF=log (10,000×g)). The chosen von Frey values are therefore equivalent to 0.45, 0.74, 1.26, 2.04, 3.31, 5.50, 8.32, and 14.45 in gram value, respectively. Starting with filament 4.31, von Frey filaments were applied perpendicularly to the ventral surface of the proximal part of the third or fourth toe for 2-3 seconds. Whenever a positive response to a stimulus occurred (paw withdrawal), the next smaller von Frey hair was applied. Whenever a negative response occurred, the next larger one was applied. The test was continued until the response of six stimuli after the first change in response had been obtained or the test had reached either end of the spectrum of the von Frey set.

The 50% threshold value was then calculated using Dixon's formula (Dixon, Ann. Rev. Phar. Tox. 20:441-462, 1980): 50% threshold=X+kd, where X is the value of the final von Frey hair used (in log units), k is the tabular value for the pattern of positive/negative responses, and d is the mean difference between stimuli in log units (0.22). In the cases where continuous positive or negative responses were observed all the way out to the end of the stimulus spectrum, values of 3.54 or 5.27 were assigned, respectively, by assuming a value of (−/+) 0.5 for k in these cases (Dixon, supra, 1980). Outcome of behavioral data were expressed as von Frey values (maximum range from 3.54 to 5.27) and plotted in a linear scale.

F. Statistical Treatment

Data were expressed as the mean±standard errors of mean (SEM). Differences in mechanical thresholds at various times after injections were tested using two-way (time and group factors) analysis of variance (ANOVA), followed by the Duncan's post-hoc test for multiple comparisons. Statistical analyses were done using the SAS® software program (SAS Institute Inc, Cary, N.C.). Two-tailed p-values<0.05 were considered to be significant.

### II. Results

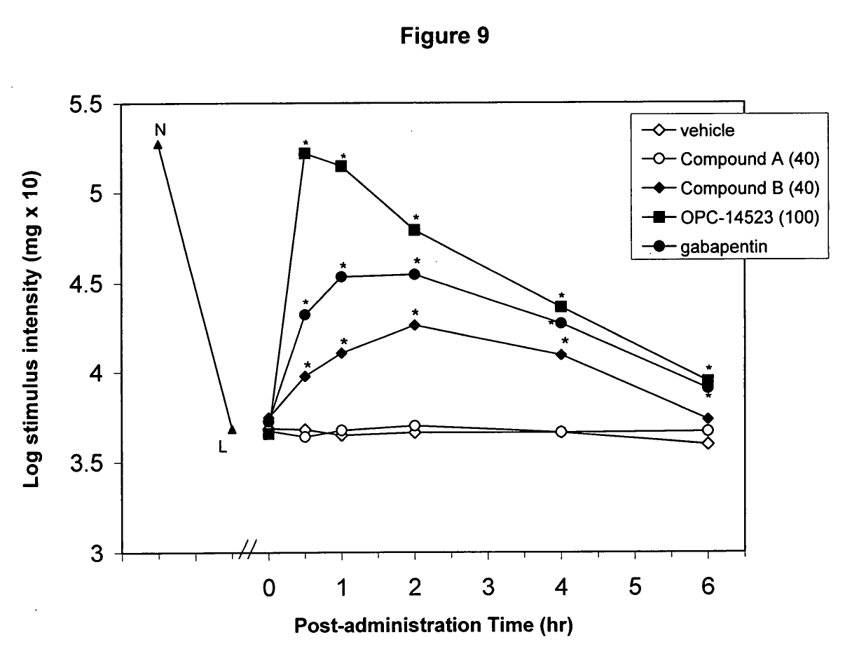

As shown in FIG. 9, mechanical thresholds 1 day before surgery (N) were greatly reduced by 4 days (L) after the ligation. Vehicle and compound A did not affect the mechanical threshold of the lesioned paw. As further shown in FIG. 9, gabapentin significantly increased the mechanical threshold of the lesioned paw for foot withdrawals over several hours. The increase induced by this dose of gabapentin was about half of the mechanical threshold observed prior to the ligation at this dose. Both compound B and OPC-14523 significantly increased the mechanical threshold of the lesioned paw for foot withdrawals over several hours. Notably, OPC-14523 increased the mechanical threshold to a level comparable to the level observed prior to ligation. These data further support the analgesic properties of OPC-14523 for neuropathic pain.

### Example 5

### Evaluation of Acute Administration of a Dose Range of OPC-14523 in a Model of Neuropathic Pain Using Tactile Stimulation and Von Frey Filaments

### I. Materials and Methods

This experiment was conducted on 8 rats in a Latin-square design examining a dose response of OPC-14523. Gabapentin was prepared at 10 mg/ml in 0.5% tragacanth and administered at 100 mg/kg p.o. Behavioral tests were conducted in a blind fashion twice (1 day and 1 hour) before as well as 1 and 4 days after the neuropathic surgery. On the fourth PO day, all rats showed similar levels of allodynic behaviors (lowering mechanical threshold for foot withdrawals). These 8 rats were randomly divided into 6 groups of 1-2 rats. Immediately after behavioral testing on the fourth PO day, rats in groups 1-6 received, respectively, an oral injection of (1) 10 mg/kg of OPC-14523, (2) 30 mg/kg of OPC-14523, (3) 60 mg/kg of OPC-14523, (4) 100 mg/kg of OPC-14523, (5) vehicle alone, and 6) 100 mg/kg of gabapentin. Behavioral tests were conducted 0.5, 1, 2, 4, and 6 hours after each injection. Then all animals rested for 1 day (or until Monday on the weekend). On the sixth PO day, each group received one of the above six compounds above that they had not received before.

The above procedure was repeated six times, so the overall experiment took 2 weeks (injections on Monday, Wednesday, Friday, and the following Monday, Wednesday, and Friday, followed by behavioral testing and 1 day of rest after each injection). The result was that all eight animals received each of the indicated six compounds in random order. Mechanical thresholds were determined in a blind fashion in that the investigator who performed the behavioral tests had no knowledge about the nature of the compounds injected. None of the rats receiving injections showed any obvious abnormal behaviors, such as motor weakness, sedation, etc.

### II. Results

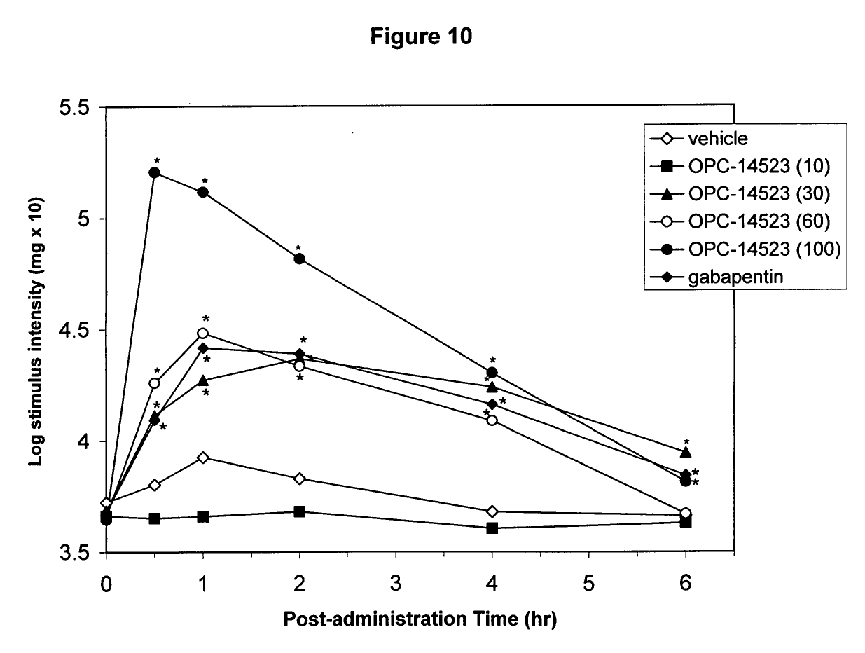

As shown in FIG. 10, neither vehicle nor OPC-14523 at 10 mg/kg produced significant changes in mechanical threshold. Gabapentin, as expected, produced a significant increase in mechanical threshold of the lesioned paw for foot withdrawals.

OPC-14523 produced a dose-dependent, statistically significant increase in mechanical threshold at 30 mg/kg, 60 mg/kg and 100 mg/kg. The level of response of OPC-14523 at 30 mg/kg and 60 mg/kg was comparable to that of gabapentin. OPC-14523 at 100 mg/kg induced an increase in mechanical threshold to about the level observed prior to the ligature surgery, exceeding the response induced by gabapentin.

These results indicate the notable analgesic properties of OPC-14523 for neuropathic pain. In this model of neuropathic pain, few treatments, surgical or pharmacological, have proven to be more effective than gabapentin. Given the known analgesic effect of gabapentin, these results indicate that OPC-14523 would have good to excellent analgesic effects in human patients.

All references cited herein are incorporated by reference. The present invention may be embodied in other specific forms without departing from the spirit or essential attributes thereof and, accordingly, reference should be made to the appended claims, rather than to the foregoing specification, as indication of the scope of the invention.

1. A method of treating pain in an individual in need of such treatment, comprising administering to the individual an effective amount of 1-[3-[4-(3-chlorophenyl)-1-piperazinyl]propyl]-5-methoxy-3,4-dihydro-2(1H)-quinolone, a prodrug thereof, or a pharmaceutically acceptable salt thereof.

2. The method according to claim 2, wherein the pharmaceutically acceptable salt is 1-[3-[4-(3-chlorophenyl)-1-piperazinyl]propyl]-5-methoxy-3,4-dihydro-2(1H)-quinolone monomethanesulfonate.

3. The method of claim 1, wherein said pain is nociceptive pain.

4. The method of claim 1, wherein said pain is pain associated with fibromyalgia.

5. The method of claim 1, wherein said pain is neuropathic pain.

6. The method of claim 5, wherein said pain is associated with a metabolic insult other than diabetes.

7. The method of claim 6 wherein said pain is associated with a systemic disorder.

8. The method of claim 5 wherein said pain is associated with a physical or chemical injury.

9. The method of claim 8 wherein said pain is associated with an injury comprising nerve root compression.

10. A method of treating fibromyalgia in an individual in need of such treatment, comprising administering to the individual an effective amount of 1-[3-[4-(3-chlorophenyl)-1-piperazinyl]propyl]-5-methoxy-3,4-dihydro-2(1H)-quinolone, a prodrug thereof, or a pharmaceutically acceptable salt thereof.

11. The method according to claim 10, wherein the pharmaceutically acceptable salt is 1-[3-[4-(3-chlorophenyl)-1-piperazinyl]propyl]-5-methoxy-3,4-dihydro-2(1H)-quinolone monomethanesulfonate.
